(19) United States
(12) Patent Application Publication
(10) Pub. No.: US 2021/0048388 A1
(43) Pub. Date: Feb. 18, 2021

(54) PORTABLE REFRACTOMETER

(71) Applicant: VALIBER LTD., Tel Aviv (IL)

(72) Inventor: Mark SHECHTERMAN, Ness Ziona (IL)

(21) Appl. No.: 16/978,926

(22) PCT Filed: Mar. 10, 2019
(86) PCT No.: PCT/IL2019/050261

§ 371 Date: Sep. 8, 2020

Related U.S. Application Data
(60) Provisional application No. 62/644,599, filed on Mar. 19, 2018.

(51) Int. Cl. G01N 21/43; G02B 5/04; G02B 3/00; G01K 13/00; G01N 33/14

(52) U.S. Cl. CPC G01N 21/431; G02B 5/04; G02B 3/00; G01K 13/00; G01N 2021/434; G02B 2003/0093; G01N 2201/0638; G01N 2201/062; G01N 2201/0612; G01N 33/143

(57) ABSTRACT

Disclosed herein, according to an aspect of some embodiments, is a dipping refractometer. The dipping refractometer includes a prism and a casing, housing a light source and a light sensor. The prism is mounted in/on the casing such as to allow dipping the prism in a fluid such that two surfaces of the prism and the fluid forming two respective direct prism-fluid interfaces. The prism, the light source, and the light sensor are configured such that for a continuous range of values of fluid refractive indices, most of the light incident on the light sensor, originating from the light source, undergoes total internal reflection off of each of the two direct prism-fluid interfaces.

### FIELD OF THE INVENTION

The invention, in some embodiments, relates to the field of refractometers and more particularly, but not exclusively, to portable refractometers for measuring refractive indices of fluids.

### BACKGROUND OF THE INVENTION

A “dipping refractometer”, also referred to in the art as an “immersion refractometer”, is a device used to measure the refractive index of a fluid. In a prism-based dipping refractometer, the immersible portion includes a prism. In operation, the immersible portion is dipped in the fluid with a face of the prism contacting the fluid such as to form therewith a prism-fluid interface. In some (prism-based) dipping refractometers, the critical angle of monochromatic light (or light having a narrow spectral distribution) incident on the prism-fluid interface is measured—e.g. by measuring the location of the light-to-shadow boundary on a reticle within the refractometer (viewable through a magnifying lens)—and the refractive index of the fluid is deduced therefrom.

State-of-the-art (prism-based) dipping refractometers may include a LED light source and a light sensor (e.g. a CCD sensor) and have dimensions similar to a hand thermometer. Some state-of-the-art digital dipping refractometers operate similarly to the dipping refractometers, described above, by measuring the location of the light-to-shadow boundary on a photodiode array.

A refractometer may be used to measure the concentration of a soluble in a fluid, as the refractive index of a fluid is dependent on the concentration of the soluble. In particular, a refractometer may be used to measure the concentration of a tastant (e.g. sugar) in a fluid. A portable refractometer may be used in the home, or even in a restaurant industry, as an aid for preparing a beverage or as a culinary aid for preparing a sauce.

U.S. Pat. No. 7,916,285 to Amamiya et al. discloses a refractometer including: a housing having an immersion portion, the immersion portion having an opening; a light source for emitting a light; a light sensor for converting a received light into an electrical signal; a prism including faces: a first face proximal to the light source and the light sensor; a second face, at least a portion of it is configured for contacting a sample liquid through the opening, and for forming an interface between the second face and the sample liquid; and a third face, wherein the light travels by the following routes: being directed towards the second face; being reflected at least in part by the interface towards the third face; and being reflected at least in part by the third face towards the light sensor. In an embodiment, the refractometer further includes a control portion for receiving the electrical signal, and for determining a refractive index of the sample liquid based at least in part on the electrical signal. In an embodiment, the control portion determines the refractive index in at least one of the following modes: a batch mode for detecting the electrical signal once and a sequential mode for detecting the electrical signal at least twice. In an embodiment, the refractometer further includes a substrate at least partially positioned within the housing, the substrate supporting the light source and the light sensor. In an embodiment, the refractometer further includes a display portion connected to the control portion for displaying a representation of the refractive index.

### SUMMARY OF THE INVENTION

Aspects of the invention, in some embodiments thereof, relate to portable refractometers. More specifically, aspects of the invention, in some embodiments thereof, relate to portable dipping refractometers.

To be accurate and reliable, a prism-based dipping refractometer generally has to be robust to several types of imperfections. The imperfections may include: (i) Penetration of light, such as daylight, from outside the prism, which travels there through and impinges on the light sensor. (ii) Fluctuations in the intensity of the light emitted by the light source, resulting from e.g. fluctuations in the driving current when the light source is a laser diode or a LED. (iii) Diffusely scattered light arriving at the light sensor. The present invention, according to some embodiments thereof, aims to address these imperfections, particularly, but not exclusively, in portable dipping refractometers.

Thus, according to an aspect of some embodiments, there is provided a dipping refractometer.

The dipping refractometer includes:
a casing, housing a light source, a light sensor, and a control unit; and
a prism including at least two exposed surfaces;

The control unit includes electronic circuitry functionally associated with the light source and the light sensor. The prism is mounted in or on the casing such as to allow dipping the prism in a fluid with the exposed surfaces and the fluid forming respective direct prism-fluid interfaces. The prism, the light source, and the light sensor, are configured such that at least some of the light emitted from the light source enters the prism, travels to one exposed surface and reflects therefrom, travels to the other exposed surface and reflects therefrom, and travels to the light sensor. The light sensor is configured to send to the control unit a signal indicative of a power of a light incident on the light sensor.

According to some embodiments of the dipping refractometer, the dipping refractometer further includes a temperature sensor configured to measure a temperature of the prism and send to the control unit a second signal indicative of the temperature of the prism.

According to some embodiments of the dipping refractometer, the prism and the temperature sensor are each mounted in or on an immersion portion of the casing. The mounting of the temperature sensor is such that the temperature sensor thermally couples to a fluid when the immersion portion is dipped in the fluid. The second signal is indicative of a temperature of the fluid, and thereby of the temperature of the prism when the prism and the fluid are in thermal equilibrium.

According to some embodiments of the dipping refractometer, the dipping refractometer further includes a reference light sensor. The prism, the light source, and the reference light sensor are configured such that some of the light emitted by the light source travels through the prism without reflecting off either of the exposed surfaces, exiting the prism such as to be incident on the reference light sensor. The reference light sensor is further configured to send to the control unit a reference signal, indicative of a power of the light incident thereon.

According to some embodiments of the dipping refractometer, substantially all the light incident on the light sensor, which originates from the light source, is reflected by both of the exposed surfaces when travelling through the prism.

According to some embodiments of the dipping refractometer, the prism includes a light entry surface where through light emitted from the light source enters the prism and where through the light incident on the light sensor exits the prism.

According to some embodiments of the dipping refractometer, the prism further includes a reflective surface including a mirror coating. The prism, the light source, and the light sensor are further configured such that light emitted from the light source, which is incident on one exposed surface, reflects from the exposed surface to the reflective surface, and reflects from the reflective surface to the other exposed surface, travelling therefrom to the light sensor.

According to some embodiments of the dipping refractometer, the reflective surface is located opposite the light entry surface, and the exposed surfaces are located opposite to one another. The exposed surfaces extend from the light entry surface to the reflective surface.

According to some embodiments of the dipping refractometer, the reflective surface is convex, being configured to function as a concave mirror with respect to light incident thereon from within the prism. The prism, the light source, and the light sensor are configured such that light exiting the prism, emitted by the light sensor and incident on the light sensor, is focused by the reflective surface such as to arrive with a small beam spread at the light sensor.

According to some embodiments of the dipping refractometer, the prism includes a rectangular prism and a spherical plano-convex lens mounted on a bottom surface of the rectangular prism. The plano-convex lens has a same refractive index as the rectangular prism. An optical axis defined by the plano-convex lens is offset relative to a longitudinal symmetry axis of the rectangular prism.

According to some embodiments of the dipping refractometer, the light source is configured to emit monochromatic light.

According to some embodiments of the dipping refractometer, the light source is configured to emit polychromatic light.

According to some embodiments of the dipping refractometer, the light source is a light-emitting diode or a laser diode.

According to some embodiments of the dipping refractometer, wherein the refractometer includes the reference light sensor and the prism further includes the reflective surface, the prism, the light source, and the reference light sensor are further configured such that the light received by the reference light sensor, which was emitted by the light source, enters the prism through the light entry surface, travels directly therefrom to the reflective surface, reflects therefrom back to the light entry surface, travelling therefrom to the reference light sensor.

According to some embodiments of the dipping refractometer, the electronic circuitry includes processing circuitry configured to determine a refractive index of a fluid, in which the refractometer is dipped, based on the signal received from the light sensor.

According to some embodiments of the dipping refractometer, the processing circuitry is configured to determine the refractive index of the fluid based also on the second signal received from the temperature sensor and/or on the reference signal received from the reference light sensor.

According to some embodiments of the dipping refractometer, the processing circuitry is configured to obtain a concentration of a tastant in the fluid from the signals received from the sensors.

According to some embodiments of the dipping refractometer, the tastant is a sweetener.

According to some embodiments of the dipping refractometer, the sweetener is sugar.

According to some embodiments of the dipping refractometer, the casing is waterproof.

According to some embodiments of the dipping refractometer, the casing is elongated, including an upper portion and an immersible lower portion, such as to allow the refractometer to be dipped within a fluid-filled drinking vessel with a user interface on the upper portion being located above the fluid. The user interface being functionally associated with the control unit.

According to some embodiments of the dipping refractometer, the user interface includes a display configured to display thereon a measured refractive index of a fluid and/or a concentration of a tastant in the fluid.

According to some embodiments of the dipping refractometer, the display is a touch screen, configured to allow a user to operate the refractometer using the touch screen.

According to some embodiments of the dipping refractometer, the dipping refractometer is further configured to display the measured concentration of a tastant in Vals.

According to some embodiments of the dipping refractometer, the control unit further includes a wireless communication interface.

According to some embodiments of the dipping refractometer, the wireless communication interface is configured to send the measured refractive index of a fluid, and/or a measured concentration of a tastant in the fluid, to an external device.

According to some embodiments of the dipping refractometer, the wireless communication unit is configured to send the signals received by the control unit from the sensors to an external device and the external device is configured to determine a refractive index of the fluid from the received signals.

According to some embodiments of the dipping refractometer, the external device is a smartphone, a smartwatch, a tablet, a personal computer, or an online server.

According to an aspect of some embodiments, there is provided a method for determining the refractive index of a fluid. The method includes the steps of:
submerging a prism in a fluid such that one or more surfaces of the prism form with the fluid one or more direct prism-fluid interfaces, respectively;
projecting a light beam into the prism;
directing at least some of the light in the light beam onto at least one of the one or more direct prism-fluid interfaces, and reflecting the light therefrom;
directing the reflected light onto at least one of the one or more direct prism-fluid interfaces, and reflecting the light therefrom;
directing the doubly-reflected light out of the prism and onto a light sensor;
converting the light arriving at the light sensor into an electrical signal indicative of the power of the arriving light; and
determining the refractive index of the fluid based on the obtained electrical signal.

According to some embodiments of the method, the method further includes a step of measuring a temperature of the prism, and, in the step of determining, the refractive index of the fluid is determined taking into account also the measured temperature of the prism.

According to some embodiments of the method, the method further includes a step of measuring a power of light in the light beam, which is not directed onto any of the direct prism-fluid interfaces, thereby recording fluctuations in the power of the light beam. In the step of determining, the refractive index of the fluid is determined taking into account also the recorded fluctuations in the power of the light beam.

According to some embodiments of the method, the light beam is monochromatic.

According to some embodiments of the method, the light beam is polychromatic.

According to an aspect of some embodiments, there is provided a dipping refractometer. The dipping refractometer includes a casing and a prism. The casing houses a light source and a light sensor. The prism is mounted in or on the casing such as to allow dipping the prism in a fluid with one or more surfaces of the prism and the fluid forming one or more direct prism-fluid interfaces, respectively. The prism, the light source, and the light sensor are configured such that for a continuous range of values of fluid refractive indices, most of the light incident on the light sensor, having travelled through the prism and originating from the light source, has undergone total internal reflection off the one or more direct prism-fluid interfaces at least twice.

According to an aspect of some embodiments, there is provided a portable dipping refractometer. The portable dipping refractometer includes:
a casing, housing a light source, a light sensor, a reference light sensor, and a control unit; and
a prism including an exposed surface.

The control unit includes electronic circuitry functionally associated with the light source, the light sensor, and the reference light sensor. The prism is mounted in or on the casing such as to allow dipping the prism in a fluid with the exposed surface and the fluid forming a direct prism-fluid interface. The refractometer is configured such as to direct a first sub-beam of a light beam, emitted by the light source into the prism, such that the first sub-beam is reflected at least partially off the exposed surface and exits the prism such as to be incident on the light sensor. The refractometer is further configured such that a second sub-beam of the light beam, emitted by the light source, is incident on the reference light sensor without having impinged on the exposed surface. The light sensor is configured to send to the control unit a signal indicative of a power of a light incident thereon, and the reference light sensor is configured to send to the control unit a reference signal indicative of a power of a light incident thereon.

According to some embodiments of the portable dipping refractometer, the refractometer further includes a mirror surface. The refractometer is further configured such that the second sub-beam enters the prism, reflects off the mirror surface, and exits the prism such as to be incident on the reference light sensor.

According to some embodiments of the portable dipping refractometer, the refractometer is further configured such that the second sub-beam is directed towards the reference light sensor without passing through the prism.

According to some embodiments of the portable dipping refractometer, the casing further includes a beam-splitter configured to receive the light beam emitted by the light source and split the light beam into the first sub-beam and the second sub-beam.

According to an aspect of some embodiments, there is provided a method for determining the refractive index of a fluid. The method includes the steps of:
submerging a prism in a fluid such that a surface of the prism forms with the fluid a to direct prism-fluid interface;
generating a light beam including a first sub-beam and a second sub-beam;
directing the first sub-beam into the prism;
directing the first sub-beam onto the direct prism-fluid interface, and reflecting the light therefrom;
directing the reflected first sub-beam light out of the prism and onto a light sensor;
converting the first sub-beam light arriving at the light sensor into a first electrical signal indicative of the power of the arriving first sub-beam light;
directing the second sub-beam onto a reference light sensor;
converting the second sub-beam light arriving at the reference light sensor into a second electrical signal indicative of the power of the second sub-beam light; and
determining the refractive index of the fluid based on the obtained electrical signals.

According to some embodiments of the method, the second sub-beam light is directed into the prism, reflected off a mirror surface of the prism, and directed therefrom onto the reference light sensor, without having impinged on the exposed surface.

Certain embodiments of the present invention may include some, all, or none of the above advantages. Further advantages may be readily apparent to those skilled in the art from the figures, descriptions, and claims included herein. Aspects and embodiments of the invention are further described in the specification hereinbelow and in the appended claims.

Unless otherwise defined, all technical and scientific terms used herein have the same meaning as commonly understood by one of ordinary skill in the art to which this invention pertains. In case of conflict, the patent specification, including definitions, governs. As used herein, the indefinite articles “a” and “an” mean “at least one” or “one or more” unless the context clearly dictates otherwise.

Embodiments of methods and/or devices herein may involve performing or completing selected tasks manually, automatically, or a combination thereof. Some embodiments are implemented with the use of components that comprise hardware, software, firmware or combinations thereof. In some embodiments, some components are general-purpose components such as general purpose computers or processors. In some embodiments, some components are dedicated or custom components such as circuits, integrated circuits or software.

For example, in some embodiments, some of an embodiment may be implemented as a plurality of software instructions executed by a data processor, for example which is part of a general-purpose or custom computer. In some embodiments, the data processor or computer may comprise volatile memory for storing instructions and/or data and/or a non-volatile storage, for example a magnetic hard-disk and/or removable media, for storing instructions and/or data. In some embodiments, implementation includes a network connection. In some embodiments, implementation includes a user interface, generally comprising one or more of input devices (e.g., allowing input of commands and/or parameters) and output devices (e.g., allowing reporting parameters of operation and results).

### BRIEF DESCRIPTION OF THE FIGURES

Some embodiments of the invention are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the invention. For the sake of clarity, some objects depicted in the figures are not to scale.

In the figures:

FIG. 1 schematically depicts a front-view of a dipping refractometer, according to some embodiments of;

FIG. 2 presents a cross-sectional view of a casing base of a casing of the dipping refractometer of FIG. 1 and of a prism mounted on the casing base, according to some embodiments;

FIG. 3 schematically depicts a side-view of the dipping refractometer of FIG. 1, disposed inside a glass with the immersion portion of the casing of the dipping refractometer being submerged in a fluid in the glass, according to some embodiments;

FIG. 4 presents a cross-sectional view of the prism and the casing base of the refractometer of FIG. 1, the prism and the casing base being submerged in a fluid, a light beam emitted from a light source in the immersion portion penetrates the prism, according to some embodiments;

FIG. 5 presents a cross-sectional view of the prism and the casing base of the refractometer of FIG. 1, the prism and the casing base being submerged in a fluid, the travel-paths within the prism of three light rays, emitted from the light sensor, are traced, according to some embodiments;

FIG. 6A presents a cross-sectional view of the prism and of the light source and a light sensor of the refractometer of FIG. 1, the prism is submerged in a fluid, travel-paths of light rays within the prism, emitted by the light source and detected by the light sensor, which undergo total internal reflection off two direct prism-fluid interfaces, are traced, according to some embodiments;

FIG. 6B presents a perspective view of the prism of the refractometer of FIG. 1, the prism is submerged in a fluid, travel-paths of light rays within the prism, emitted by the light source and detected by the light sensor, which undergo total internal reflection off two direct prism-fluid interfaces, are traced, according to some embodiments;

FIG. 7A presents a cross-sectional view of the prism and of the light source and a reference light sensor of the refractometer of FIG. 1, the prism is submerged in a fluid, travel-paths of light rays within the prism, emitted by the light source and detected by the reference light sensor, which are not incident on any direct prism-fluid interface, are traced, according to some embodiments;

FIG. 7B presents a perspective view of the prism of the refractometer of FIG. 1, the prism is submerged in a fluid, travel-paths of light rays within the prism, emitted by the light source and detected by the reference light sensor, which are not incident on any direct prism-fluid interface, are traced, according to some embodiments;

FIG. 8A presents a cross-sectional view of the prism and of the light source, light sensor, and reference light sensor of the refractometer of FIG. 1, the prism is submerged in a fluid, the light ray travel-paths from FIG. 6A and the light ray travel paths from FIG. 7A are both traced, according to some embodiments;

FIG. 8B presents a perspective view of the prism of the refractometer of FIG. 1, the prism is submerged in a fluid, the light ray travel-paths from FIG. 6B and the light ray travel paths from FIG. 7B, are both traced, according to some embodiments;

FIGS. 9A-9F presents a cross-sectional view of the prism of the refractometer of FIG. 1, while the prism is submerged in a fluid. Travel-paths of light rays within the prism are emitted by the light source and detected by the light sensor, said light rays undergo a total internal reflection off two direct prism-fluid interfaces and traced, for six different values of the refractive index of the fluid, according to some embodiments;

FIG. 10 presents a block diagram of the dipping refractometer of FIG. 1, according to some embodiments;

FIG. 11 compares the normalized power of measured light using the refractometer of FIG. 1 and an alternative refractometer identical to the refractometer of FIG. 1 except for including a single direct prism-fluid interface instead of two direct prism-fluid interfaces, according to some embodiments;

FIG. 12 presents a cross-sectional view of an immersion portion and a prism of a dipping refractometer, according to some embodiments; and

FIG. 13 presents a block diagram of a dipping refractometer, according to some embodiments.

### DETAILED DESCRIPTION OF SOME EMBODIMENTS

The principles, uses and implementations of the teachings herein may be better understood with reference to the accompanying description and figures. Upon perusal of the description and figures present herein, one skilled in the art is able to implement the teachings herein without undue effort or experimentation. In the figures, same reference numerals refer to same parts, respectively, throughout.

As used herein, a “surface”, such as a surface of a prism, can refer to a single flat or curved surface, as well as to a number of adjacent surfaces which are “sharply joined”, such as a number of adjacent faces of a polytope (e.g. two faces of a cube having a common edge).

As used herein, a non-coated prism surface immersed in a fluid forms therewith a “direct prism-fluid interface”. In contrast, a coated prism surface immersed in a fluid—such that the coating has a refractive index which differs, or differs substantially, from both the refractive index of the prism and the refractive index of the fluid—does not form therewith a “direct prism-fluid interface”.

As used herein, according to some embodiments, a range of refractive indices between which a refractometer can distinguish (up to the measurement resolution thereof) is referred to as the “measurement range” of the refractometer.

As used herein, “light” refers to electromagnetic radiation, including, but not limited to, visible light (electromagnetic radiation characterized by wavelengths from about 390 nm to about 700 nm), infrared light, and ultraviolet light.

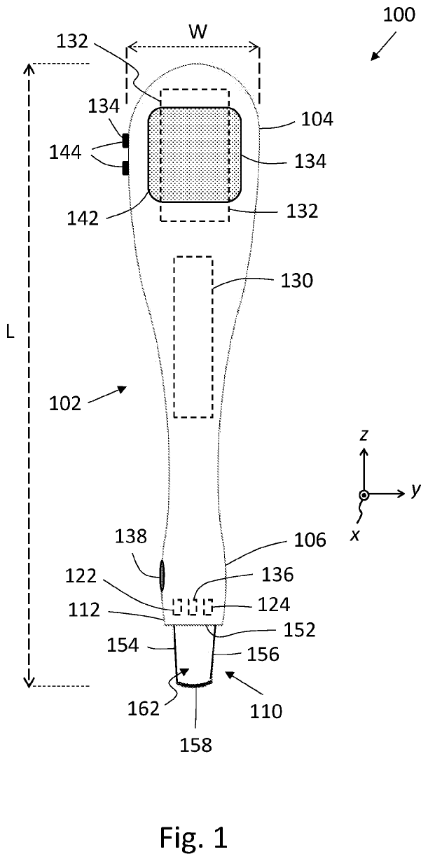

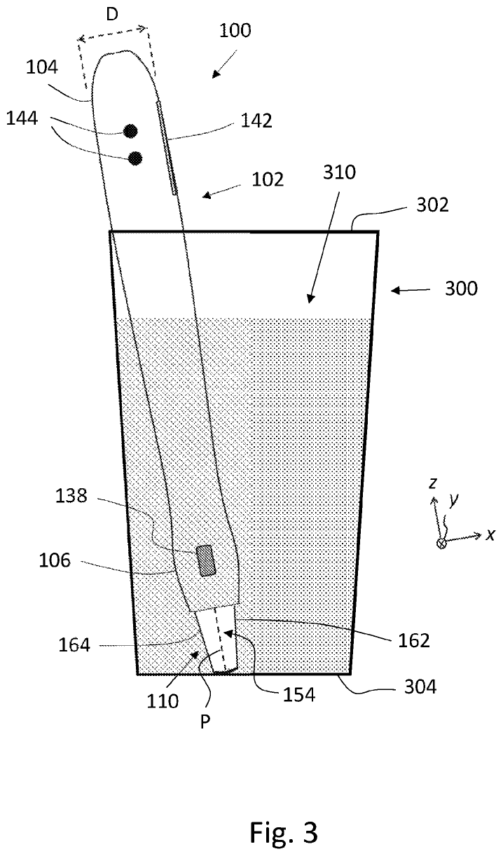

FIG. 1 schematically depicts a front view of a dipping refractometer 100, according to some embodiments disclosed herein. Refractometer 100 is elongated, having dimensions similar to a pen or a (clinical) hand thermometer, e.g. refractometer 100 may have a length L (i.e. height) measuring between about 8 cm to about 15 cm, a width W measuring between about 2 cm to about 5 cm, and a depth (thickness) D (indicated in FIG. 3) measuring between about 0.5 cm to about 1.5 cm. Refractometer 100 includes a casing 102, having an upper portion 104 and an immersion portion 106, which constitutes a lower portion of casing 102. Refractometer 100 further includes a prism 110 mounted in/on immersion portion 106 at a casing base 112 of casing 102, as elaborated on below. According to some embodiments, prism 110 measures between about 0.5 cm to about 1.5 cm in height, and between about 0.3 cm to about 0.8 cm in width and similarly in depth. Casing 102 includes, housed therein, a light source 122, a light sensor 124, a control unit 130, and a battery 132 for powering refractometer 100 operation. Casing 102 further includes a reference sensor 136 housed in immersion portion 106. According to some embodiments, casing 102 further includes a user interface 134 on upper portion 104 and/or a temperature sensor 138 mounted in/on immersion portion 106. Light source 122, sensors 124, 136, and 138, control unit 130, and battery 132 are all not visible from outside of casing 102, and as such are delineated by dashed lines.

To facilitate the description, in some of the figures a three-dimensional Cartesian coordinate system is depicted. In each of the figures the orientation of the coordinate system is such that the direction defined by refractometer 100 length L is parallel to the z-axis, the direction defined by refractometer 100 width W is parallel to the y-axis, and the direction defined by refractometer 100 depth D is parallel to the x-axis.

Immersion portion 106 is waterproof. According to some embodiments, casing 102 is waterproof, thereby allowing washing refractometer 100, e.g. under a tap. Immersion portion 106 may be made of a corrosion-resistant material—such as stainless steel or plastics including polyvinyl chloride (PVC), polytetrafluoroethylene (PTFE), polyethylene (PE), and polypropylene (PP)—thereby allowing for the use/repeated use of refractometer 100 in acidic and caustic fluids (e.g. beverages such as cola and ginger tea, respectively, when used in the home as a cooking aid, or acidic fluids and alkaline fluids, respectively, when used in the lab).

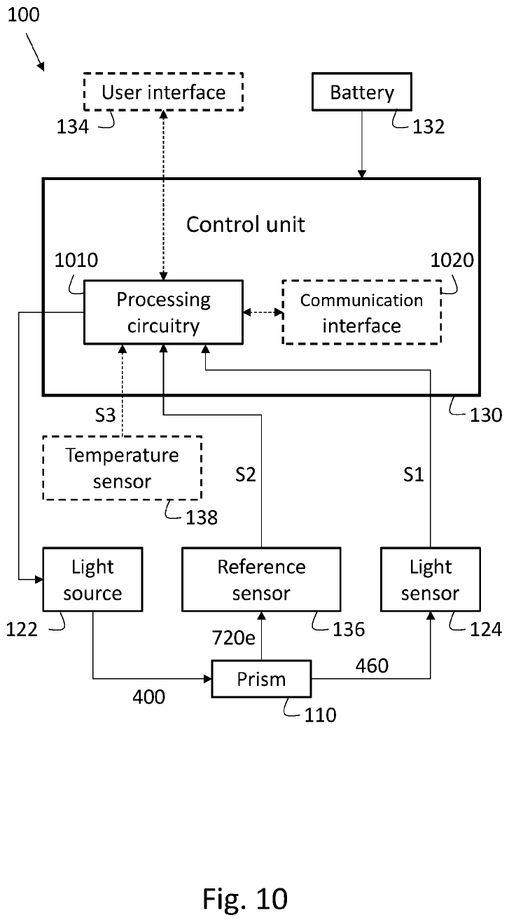

Control unit 130 includes electronic circuitry (e.g. processing circuitry, amplifying circuitry, analog-to-digital (A/D) conversion circuitry) and is configured to control refractometer 100 operation, as elaborated on below and in the description of FIG. 10. Light source 122 is configured to emit a light beam directed at prism 110, as elaborated on below. According to some embodiments, the light beam is monochromatic (e.g. when light source 122 is a laser diode). According to some embodiments, the light beam is polychromatic (e.g. when light source 122 is a light emitting diode (LED). Light sensor 124 is configured detect light incident thereon, and to send to control unit 130 an electrical signal S1 indicative of the power of the incident light. Light sensor 124 may be, for example, a charge-coupled device (CCD) sensor or a complementary metal-oxide semiconductor (CMOS) sensor, or may include a phototransistor, as further elaborated on below. Similarly, reference sensor 136 is configured detect light incident thereon, and to send to control unit 130 a reference (electrical) signal S2 indicative of the power of the incident light. Reference sensor 136 may be, for example, a charge-coupled device (CCD) sensor or a complementary metal-oxide semiconductor (CMOS) sensor, or may include a phototransistor.

As used herein, according to some embodiments, “polychromatic light beam” can refer to a light beam having a continuous spectral distribution, as well as to a light beam including two or more monochromatic light beams of different wavelength.

Temperature sensor 138 is mounted in/on immersion portion 106 such as to thermally couple to a fluid in which immersion portion 106 is submerged. For example, as depicted in the figures, temperature sensor 138 may include an exposed portion (not numbered) which comes into direct contact with the fluid when immersion portion 106 is submerged therein.

Temperature sensor 138 is configured to send to control unit 130 an electrical signal S3 indicative of the temperature of the fluid. Temperature sensor 138 may be, for example, a thermocouple or a resistance temperature detector (RTD). It is noted that signal S3 will also be indicative of the temperature of prism 110, once the fluid and prism 110 reach thermal equilibrium. The skilled person will appreciate that other embodiments and/or configurations of temperature sensors are possible for measuring the temperature of prism 110. For example, according to some embodiments (not depicted in the figures), immersion portion 106 houses a temperature sensor (in place of, or in addition to, temperature sensor 138), which is directly thermally coupled to prism 110, or thermally coupled thereto via a heat-conducting element.

Control unit 130 is configured to process the electrical signals received from sensors 124, 136 (and the temperature sensor in embodiments wherein refractometer 100 includes a temperature sensor) to obtain a measured value of a refractive index n_{f} of the fluid and, according to some embodiments, the measured value of the concentration of a tastant, such as sugar or salt, in the fluid, as elaborated on below.

User interface 134 includes a display 142. Display 142 may be a LED-based display, a liquid-crystal display (LCD), or the like. Display 142 is configured to receive processed measurement data from control unit 130 and to display the received data. The received data may include the measured value of n_{f} and/or optionally the measured concentrations of one or more tastants. According to some embodiments, display 142 is a touch screen, thereby allowing a user to control the operation of refractometer 100 (e.g. to switch on refractometer 100, to instruct control unit 130 to initiate measurement of n_{f}) by means of the touch screen. According to some embodiments, user interface 134 may include alternative/additional input means, for example, in the form of buttons 144, as shown in the figures.

Battery 132 is disposed within a battery compartment (not shown). Casing 102 may include a removable battery compartment cover (not shown), thereby allowing to replace battery 132.

According to some embodiments, battery 132 is rechargeable and casing 102 includes a port (not shown) for recharging battery 132. According to some embodiments, battery 132 may be recharged wirelessly.

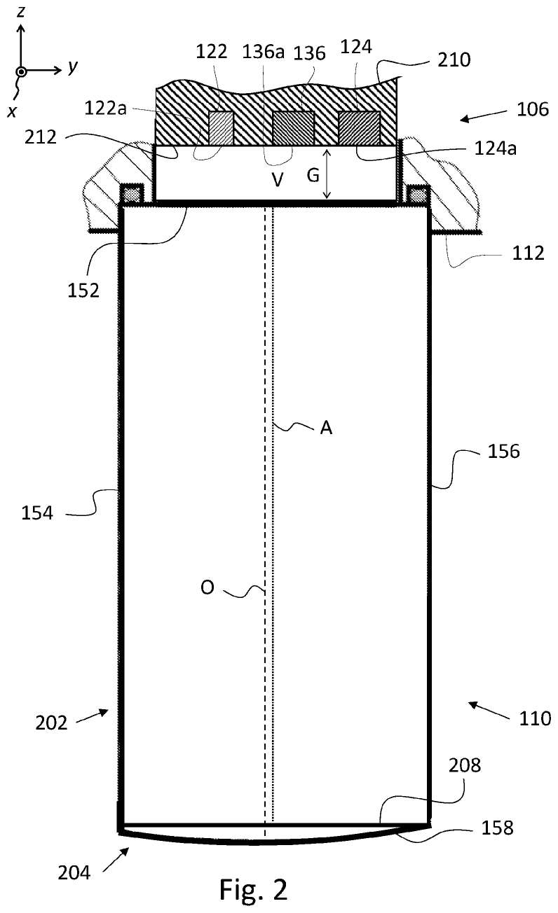

Prism 110 includes a plurality of surfaces with at least one of the plurality of surfaces being exposed or partially exposed, such as to form a direct prism-fluid interface when refractometer 100 is dipped in a fluid. Making reference also to FIG. 2, according to some embodiments, prism 110 includes four surfaces: a first surface 152, a second surface 154, a third surface 156 opposite second surface 154, and a fourth surface 158 opposite first surface 152. Second surface 154 and third surface 156 each extend from first surface 152 to fourth surface 158. Prism 110 exhibits reflection symmetry about a (flat) plane P (indicated in FIG. 3). Plane P extends parallel to the yz-plane, such as to bisect prism 110. According to some embodiments, second surface 154 and third surface 156 are flat and parallel or substantially parallel. Two additional surfaces, a fifth surface 162 and a sixth surface 164 (shown in FIG. 3), are parallel or substantially parallel to one another (and to plane P) and substantially parallel to second surface 154 and third surface 156, each of fifth surface 162 and sixth surface 164 extending from first surface 152 to fourth surface 158. Fourth surface 158 includes a reflective coating, e.g. is coated by a mirror coating, which can be, for example, a metal or a multi-layer dielectric reflecting coating.

As used herein, according to some embodiments, “exposed surface” and “partially exposed surface” are used interchangeably.

The skilled person will appreciate that other geometries of prism 110 may apply. For example, prism 110 may have a round transverse cross-section (i.e. perpendicularly to a longitudinal symmetry axis of prism 110), or second surface 154 and third surface 156 (as well as fifth surface 162 and sixth surface 164) may be centrally inclined from first surface 152 towards fourth surface 158.

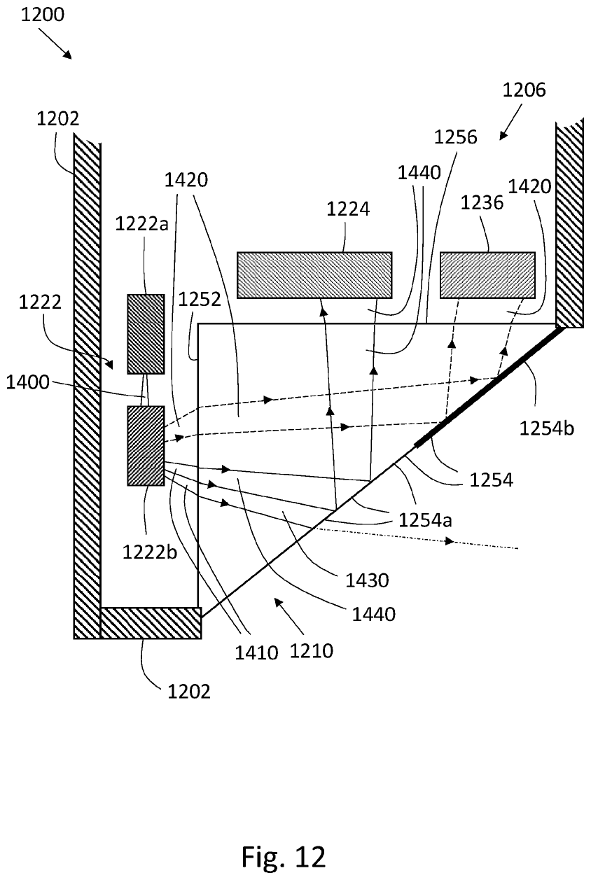

The skilled person will also appreciate that the use of a reference sensor, such as reference sensor 136, is not limited to a dipping refractometer including a prism which forms two or more direct prism-fluid interfaces when the refractometer is dipped in a fluid. The scope of the disclosed technology also covers refractometers including two light sensors, such as sensors 124 and 136, and a prism, which forms only one direct prism-fluid interface when the refractometer is dipped in a fluid. Thus, according to some embodiments, there is provided a refractometer similar to refractometer 100. The refractometer differs from refractometer 100 in including a prism which differs from refractometer 100 embodiments, depicted in the figures, in having one of the second and third surfaces thereof (i.e. the surfaces corresponding to second surface 154 and third surface 156, respectively, in prism 110) coated by a mirror coating (so that only the non-coated surface forms a direct prism-fluid interface with a fluid when the refractometer is dipped in the fluid). Other examples of refractometers covered by the disclosed technology include refractometers with two light sensors and a triangular prism having a single exposed surface, as elaborated on below in the description of FIG. 12.

FIG. 2 presents a cross-sectional view of prism 110 and a lower part of immersion portion 106 taken along plane P. According to some embodiments, fourth surface 158 is convex. That is to say, the bottom of prism 110 is convex, so that fourth surface 158 constitutes a concave mirror with respect to light incident thereon from within prism 110. According to some such embodiments, fourth surface 158 is spherical, thereby constituting a (concave) spherical mirror with respect to light incident thereon from within prism 110.

For example, prism 110 may be formed of a rectangular prism 202 and a plano-convex lens 204 made of the same material as rectangular prism 202 and coated with a mirror coating. Plano-convex lens 204 is mounted on a bottom surface 208 of rectangular prism 202, such that the convex surface of plano-convex lens 204 constitutes fourth surface 158 (of prism 110). A longitudinal symmetry axis A of rectangular prism 202 is defined by a central axis—extending along the length of rectangular prism 202 (i.e. parallel to the z-axis)—about which rectangular prism 202 exhibits symmetry under one or more rotations by 90°. An optical axis O of prism 110 extends along the length thereof (i.e. parallel to the z-axis), passing through both the bottommost point (not indicated) of plano-convex lens 204 and the center of curvature (not indicated) of plano-convex lens 204. For example, when plano-convex lens 204 is a spherical mirror, optical axis O is normal to the mirror surface at the vertex of the mirror surface. The center of curvature may be located outside of prism 110 (on an extension of plane P into immersion portion 106), e.g. slightly above light source 122 and sensors 124 and 136, as elaborated on below. According to some embodiments, as depicted in FIG. 2, optical axis O is offset (on plane P) with respect to longitudinal symmetry axis A, towards second surface 154. (Both optical axis O and longitudinal symmetry axis A lie on plane P.) That is to say, optical axis O and longitudinal symmetry axis A do not coincide (but are parallel), as elaborated on below. According to some embodiments, optical axis O and longitudinal symmetry axis A coincide.

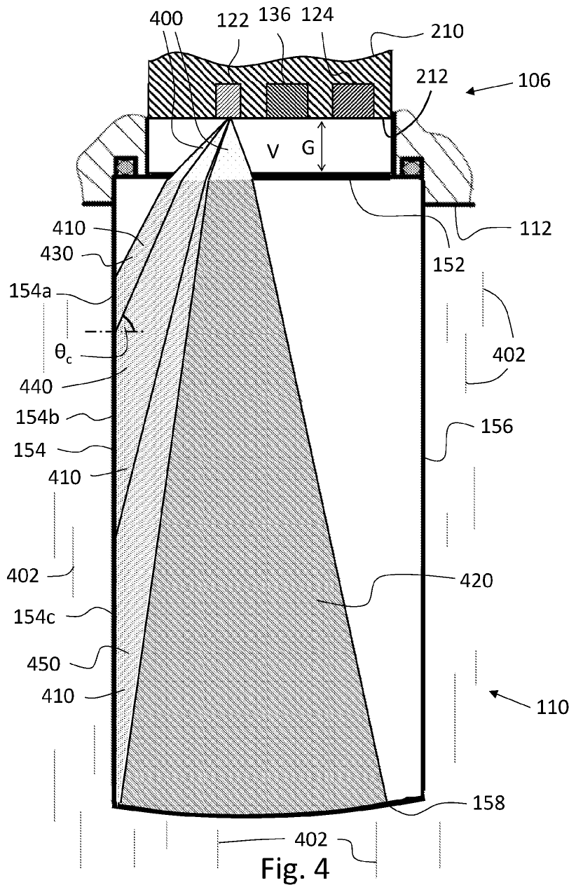

First surface 152 is embedded in/attached to casing base 112. According to some embodiments, first surface 152 is exposed inside an inner volume V within immersion portion 106, forming a (direct) prism-air interface with air in inner volume V. For example, casing base 112 may include an opening at the bottom thereof adapted to the perimeter (not numbered) of prism 110 at the region below first surface 152. The attachment may be fluidly sealed, for example, by means of a gasket or a sealing glue. The prism-air interface defines a critical angle φ_{c} (which is dependent on the wavelength of the incident light; φ_{c} is not indicated in the figures). Second surface 154 and third surface 156 are at least partially exposed (outside of immersion portion 106) and non-coated. The mounting of prism 110 in/on casing base 112 allows dipping prism 110 in a fluid, with second surface 154 forming a direct prism-fluid interface with the fluid and with third surface 156 forming a (second) direct prism-fluid interface with the fluid, as shown in FIGS. 3-4. The direct prism-fluid interfaces define a critical angle θ_{c} (which is dependent on the wavelength of the incident light; θ_{c} is indicated in FIG. 4).

Light source 122, light sensor 124, and reference sensor 136 are positioned within immersion portion 106 above first surface 152. Reference sensor 136 is positioned between light source 122 and light sensor 124. Each of a light-emitting portion 122a of light source 122, a light-sensing surface 124a of light sensor 124, and a light-sensing surface 136a of reference sensor 136 is oriented facing first surface 152. According to some embodiments, casing 102 includes, mounted therein, a substrate 210. Substrate 210 is substantially flat, extending along plane P inside immersion portion 106. A substrate bottom edge 212 extends parallel to, and proximately to, first surface 152, with inner volume V forming a gap G there between. Light source 122, light sensor 124, and reference sensor 136 are mounted at substrate bottom edge 212. According to some embodiments, substrate 210 is a printed circuit board (PCB), extending within casing 102 from immersion portion 106 to upper portion 104. Control unit 130 is mounted on the PCB above light source 122 and sensors 124 and 136.

FIG. 3 schematically depicts a side-view of refractometer 100 dipped in a (drinking) glass 300. Glass 300 includes a glass rim 302 and a glass bottom 304. Glass 300 is partially filled with a fluid 310. Refractometer 100 is partially disposed in glass 300 with upper portion 104 (of casing 102) and fourth surface 158 (of prism 110) resting against glass rim 302 and glass bottom 304, respectively, thereby supporting refractometer 100. Refractometer 100 is dipped in fluid 310, with immersion portion 106 and prism 110 being fully submerged in fluid 310.

In operation, refractometer 100 is dipped in a vessel, such as the drinking vessel depicted in FIG. 3 or a cooking pot, filled/partially filled with a fluid, with prism 110 submerged in the fluid. FIG. 4 presents a cross-sectional view of prism 110, and a lower part of immersion portion 106 taken along plane P. Immersion portion 106 (and prism 110) is submerged in a fluid, the fluid being indicated by vertical lines 402. A light beam 400, emitted from light source 122, enters prism 110 through first surface 152. To facilitate the description, light beam 400 is assumed to be either monochromatic or polychromatic with a narrow spectral width, in which case θ_{c} is substantially constant over the spectral width. Nevertheless, as explained below, refractometer 100 may also be used to measure the refractive index of a fluid also when light source 122 emits a light beam having a broad spectral width or when light source 122 emits a number of monochromatic light beams.

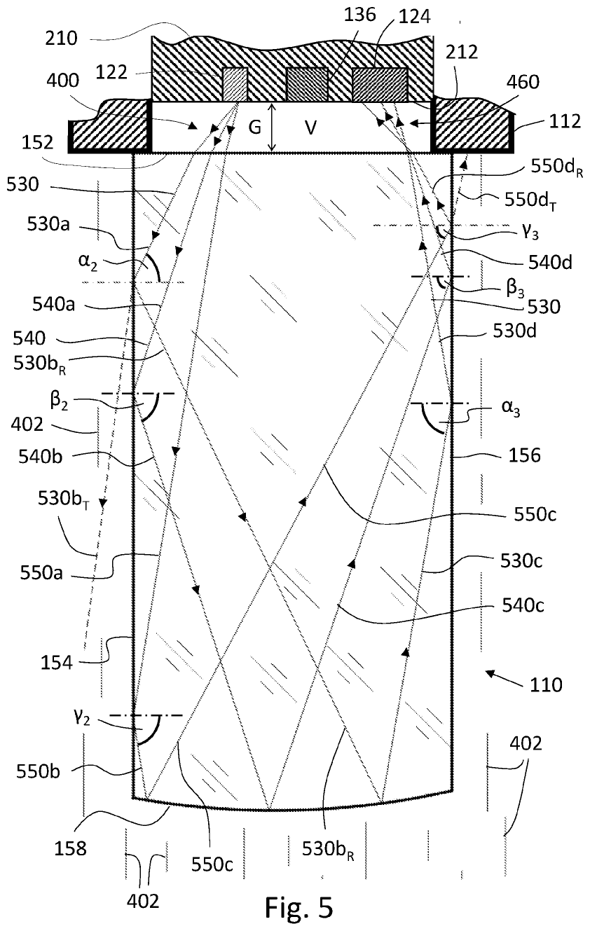

The entering light beam includes two light sub-beams, (which may be adjacent, as depicted in the figures): a first sub-beam 410 and a second sub-beam 420. First sub-beam 410 includes three adjacent sub-beam portions: a first sub-beam portion 430, a second sub-beam portion 440 adjacent to first sub-beam portion 430, and a third sub-beam portion 450 adjacent to second sub-beam portion 440. Light rays in first sub-beam portion 430 are incident on second surface 154 at an angle smaller than θ_{c} and are only partially reflected (i.e. each of the light rays separates into a refracted light ray (as shown in FIG. 5) which emerges into the fluid and a reflected light ray (as shown in FIG. 5)). Light rays in second sub-beam portion 440 and third sub-beam portion 450 are incident on second surface 154 at an angle greater than θ_{c} and undergo total internal reflection (TIR), that is to say, are fully reflected from second surface 154 (as shown in FIG. 5).

A first incidence area 154a , a second incidence area 154b , and a third incidence area 154c on second surface 154 indicate areas on second surface 154 whereon first sub-beam portion 430, second sub-beam portion 440, and third sub-beam portion 450 are incident, respectively. It is noted that the sizes of incidence areas 154a and 154c increase with n_{f}, as explained below. This increase comes at the expense of the size of second incidence area 154b , due to the increase in the value of the critical angle θ_{c} (for creating total internal reflection (TIR)).

More specifically, the path within prism 110 of an arbitrary light ray 530, travelling on plane P and originating from first sub-beam portion 430, is traced in FIG. 5. (Prism 110 is submerged in the fluid indicated by vertical lines 402.) The path of light ray 530 is divided into four (straight) travel lines, associated with four respective light rays (which make up light ray 530): a light ray 530a , a light ray 530b_{R}, a light ray 530c , and a light ray 530d . Light ray 530a travels from first surface 152 to second surface 154. Light ray 530a is incident on second surface 154 at an angle az <O. Consequently, light ray 530a is partially reflected off second surface 154, separating into reflected light ray—light ray 530b_{R}—and a refracted (transmitted) light ray 530b_{T}, which emerges from prism 110 into the fluid. The (partially) reflected light ray, light ray 530b R, is directed towards fourth surface 158 and is fully reflected therefrom—indicated by light ray 530c —towards third surface 156. Light ray 530c is incident on third surface 156 at an angle α₃≥θ_{c}. Consequently, light ray 530c undergoes TIR (total internal reflection) off third surface 156. The reflected light ray, light ray 530d , is directed towards first surface 152. Since light ray 530d incidence angle on first surface 152 is significantly smaller than the critical angle (p_{c}, defined by the (direct) prism-air interface (formed by prism 110 and air within inner volume V), almost all of light ray 530d exits from prism 110.

Similarly, the path within prism 110 of an arbitrary light ray 540, travelling on plane P and originating from second sub-beam portion 440, is also traced in FIG. 5. The path of light ray 540 is divided into four (straight) travel lines, associated with four respective light rays (which make up light ray 540): a light ray 540a , a light ray 540b , a light ray 540c , and a light ray 540d . Light ray 540a travels from first surface 152 to second surface 154. Light ray 540a is incident on second surface 154 at an angle β₂≤θ_{c}. Consequently, light ray 540a undergoes TIR off second surface 154. The reflected light ray, light ray 540b , is directed towards fourth surface 158 and is fully reflected therefrom—indicated by light ray 540c —towards third surface 156. Light ray 540c is incident on third surface 156 at an angle β₃≥θ_{c}(β₃<α₃). Consequently, light ray 540c undergoes TIR off third surface 156. The reflected light ray, light ray 540d , is directed towards first surface 152. Since light ray 540d incidence angle on first surface 152 is significantly smaller than (p_{c}, almost all of light ray 540d exits from prism 110.

Finally, the path within prism 110 of an arbitrary light ray 550, travelling on plane P and originating in third sub-beam portion 450, is also traced in FIG. 5. The path of light ray 550 is divided into four (straight) travel lines, associated with four respective light rays (which make up light ray 550): a light ray 550a , a light ray 550b , a light ray 550c , and a light ray 550d_{R}. Light ray 550a travels from first surface 152 to second surface 154. Light ray 550a is incident on second surface 154 at an angle γ₂≥θ_{c}(γ₂≥β₂). Consequently, light ray 550a undergoes TIR off second surface 154. The reflected light ray, light ray 550b , is directed towards fourth surface 158 and is fully reflected therefrom, indicated by light ray 550c , towards third surface 156. Light ray 550c is incident on third surface 156 at an angle γ₃<θ_{c}(γ₃<β₃). Consequently, light ray 550c is partially reflected off of third surface 156, separating into reflected light ray—light ray 550d_{R}—and a refracted (transmitted) light ray 550d_{T}, which emerges from prism 110 into the fluid. Light ray 550d_{R} is directed towards first surface 152. Since light ray 550d_{R} incidence angle on first surface 152 is significantly smaller than the critical angle φ_{c}, almost all of light ray 550d_{R} exits from prism 110.

It is noted that prism 110, light source 122, and light sensor 124 are configured such that substantially every light ray, originating from first sub-beam 410 and which arrives at light sensor 124, will undergo TIR off a direct prism-fluid at least once before arriving at light sensor 124:
Light rays in first sub-beam portion 430 partially reflect off second surface 154 and undergo TIR off third surface 156.
Light rays in second sub-beam portion 440 undergo TIR off both second surface 154 and third surface 156.
Light rays in third sub-beam portion 450 undergo TIR off second surface 154 and partially reflect off third surface 156.

That is to say, prism 110, light source 122, and light sensor 124 relative positions, and prism 110 geometry are such that substantially every light ray impinging on light sensor 124, which can be traced back to first sub-beam 410, undergoes TIR off second surface 154 and/or third surface 156. Except for n_{f} values close to the top of refractometer 100 measurement range, at which the size of incidence area 154b is very small and consequently the power of second sub-beam portion 440 is very small, the bulk of the contribution to the light incident on light sensor 124 arises from light rays which can be traced back to second sub-beam portion 440, i.e. light rays that undergo TIR twice (off direct prism-fluid interfaces) before arriving at light sensor 124.

Light beam 460 (indicated also in FIG. 10) is made up of light rays originating from sub-beam portions 430, 440, and 450 which are incident on light sensor 124. Except for when n_{f} is close to the top of refractometer 100 measurement range, light beam 460 is mainly made up of light rays originating from second sub-beam portion 440.

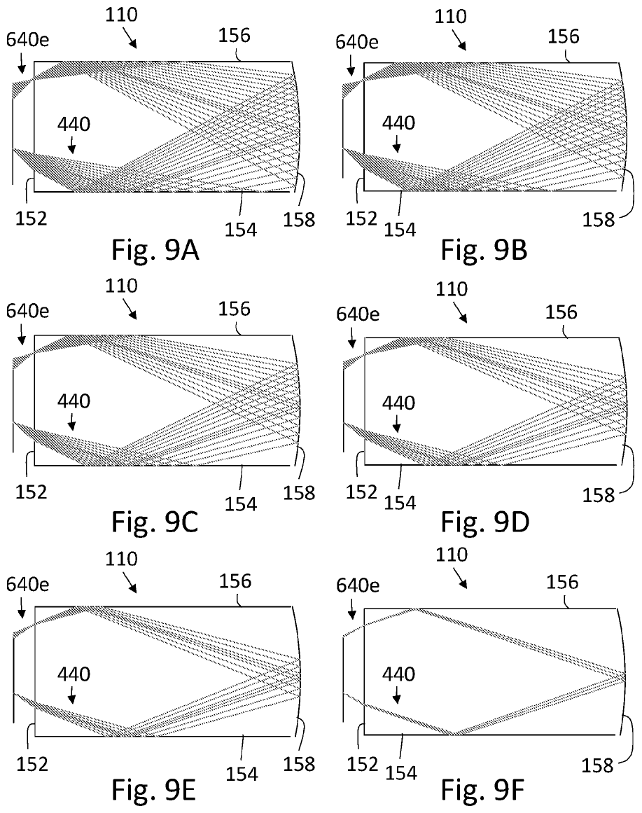

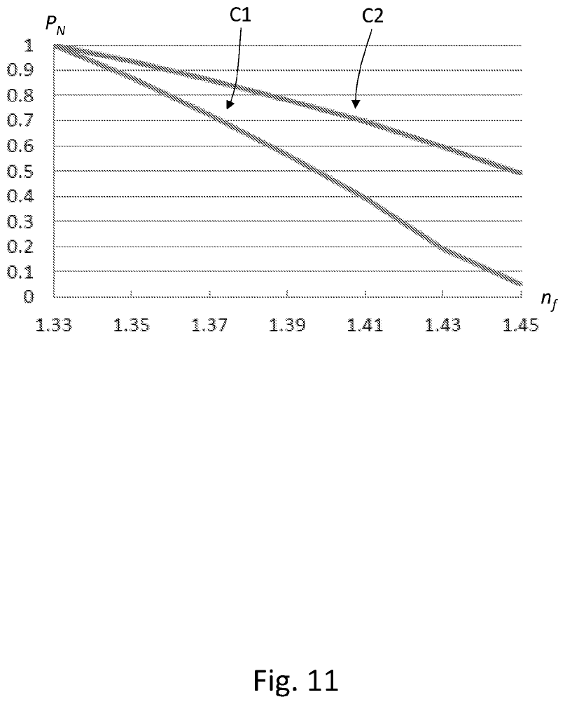

Refractometer 100 characterizing parameters, such as the geometry of prism 110 (e.g. the length and width thereof, the radius of curvature of fourth surface 158), the refractive index of prism 110, the width of gap G, the wavelength of the light emitted by light source 122 and the numerical aperture of light beam 400, may be selected based on the desired measurement range of n_{f}. Specific examples of refractometer 100 characterizing parameters and the respective corresponding measurement ranges of n_{f} are specified below in the descriptions of FIGS. 9A-9F and FIG. 11.

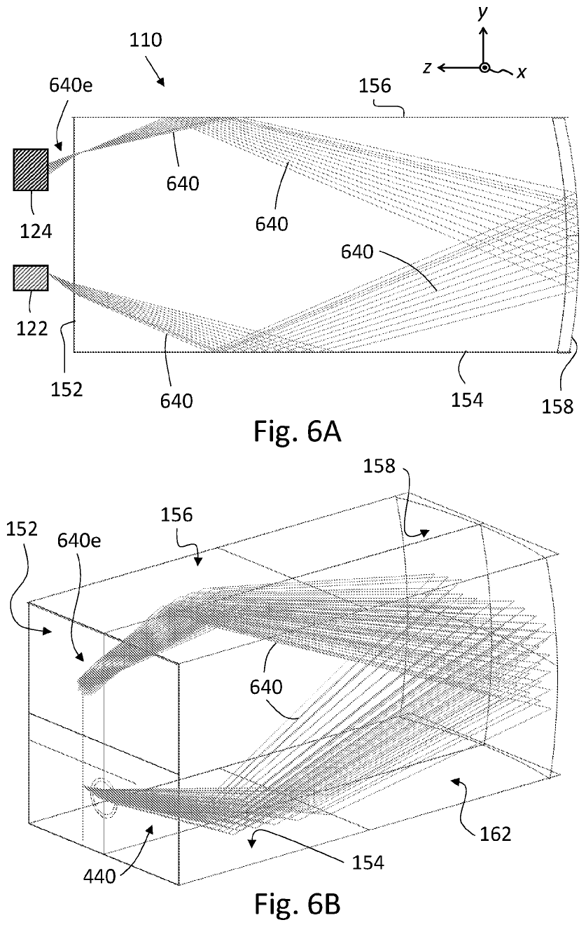

FIG. 6A presents a cross-sectional view of prism 110 taken along plane P. Prism 110 is submerged in a fluid (not depicted). Light source 122 and light sensor 124 are also shown. Light from second sub-beam portion 440, indicated by light rays 640 (light ray 540 in FIG. 5 is one of light rays 640), travels from first surface 152 to second surface 154, and fully reflects towards fourth surface 158. The light incident on fourth surface 158 is (fully) reflected towards third surface 156. The light incident on third surface 156 is fully reflected towards first surface 152. Since the respective angle of incidence on first surface 152 of substantially every one of light rays 640 is significantly smaller than (p_{c}, substantially all of light rays 640 exit prism 110 through first surface 152. Due to the magnitude of fourth surface 158 radius of curvature being close to, or approximately equal to, the optical path length from light source 122 to the fourth surface 158 (through reflection from second surface 154), fourth surface 158 focuses the light reflected therefrom, such that light rays 640 arrive close to focused at first surface 152 (after reflecting from third surface 156). Specifically, exiting light rays 640e will be fully focused on light sensor 124, when the optical path length from light source 122 to fourth surface 158 is exactly equal to the optical path length from fourth surface 158 to light sensor 124. Consequently, the exiting light, indicated by light rays 640e , arrives at light sensor 124 with a small beam spread and high intensity (power per unit area perpendicular to the travel direction of the light).

High intensity increases the signal-to-noise ratio (by allowing light sensing surface 124a to be accordingly small, thereby registering less “noise”). In particular, the high intensity may help to offset the contribution of scattered light (e.g. light diffusely reflected off one or more of surfaces 154, 156, 158, 162, and 164)—as well as light not from light source 122 which enters prism 110 (e.g. daylight and/or light from light fixtures)—to the obtained signal S1. It is noted that when optical axis O is offset towards second surface 154 with respect to longitudinal symmetry axis A, the exiting light rays will focus before arriving at light sensor 124. The off-setting of optical axis O (relative to longitudinal symmetry axis A) shifts light rays 640e onto light sensor 124. Specifically—due to optical axis O off-setting—light source 122 and light sensor 124 are not positioned symmetrically with respect to longitudinal symmetry axis A (nor with respect to optical axis O), as elaborated on below in the description of FIGS. 9A-9F.

According to some embodiments, immersion portion 106 further includes a pair of optical filters (not shown) positioned in inner volume V below light sensor 124 and reference sensor 136, respectively. The optical filters have high transmission for the portion of the optical spectrum corresponding to light source 122 emission and low transmission for the rest of the optical spectrum. The optical filters can decrease light-noise (i.e. increase signal-to-noise-ratio) and parasitic “ghost light” from external light sources (e.g. sunlight, light from light fixtures, such as lamps).

To facilitate the description, in FIG. 6A, only light rays on plane P are indicated. However, the spread of light beam 400 and the spread of second sub-beam portion 440 (as well as the respective spreads of sub-beam portions 430 and 450, and second sub-beam 420) are not confined to plane P. FIG. 6B presents a perspective view of prism 110. (Prism 110 is submerged in a fluid (not depicted). Light source 122 and light sensor 124 are not shown.) The spread of second sub-beam portion 440 within prism 110 (beyond plane P), and travel-paths of light rays 640 within prism 110 (also outside of plane P), are depicted.

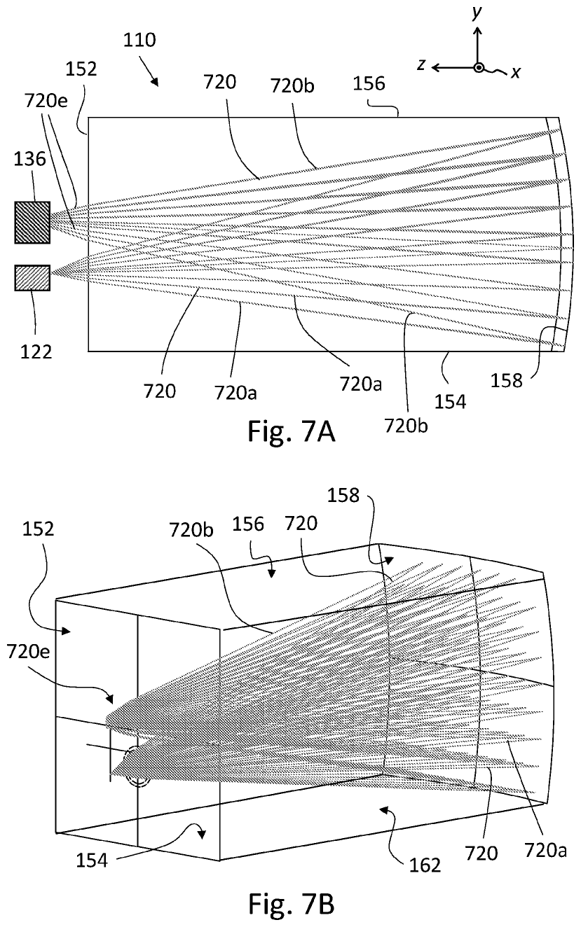

FIG. 7A presents a cross-sectional view of prism 110 taken along plane P. Prism 110 is submerged in a fluid (not depicted). Light source 122 and reference sensor 136 are also shown. The paths within prism 110 of light rays 720, travelling on plane P and originating from second sub-beam 420, are traced. The path of each of light rays 720 is divided into two respective (straight) travel lines, associated with two respective light rays: a light ray 720a and a light ray 720b . Light rays 720a travel directly from first surface 152 to fourth surface 158 (i.e. without being reflected along the way off either or both of second surface 154 and third surface 156). Light rays 720a are fully reflected off fourth surface 158. The reflected light rays, light rays 720b , travel directly therefrom back to first surface 152. Since the respective angle of incidence on first surface 152 of substantially every one of light rays 720b is significantly smaller than (p_{c}, substantially all of light rays 720b exit prism 110 through first surface 152. Due to fourth surface 158 radius of curvature being slightly greater than the length of prism 110, fourth surface 158 focuses the light reflected therefrom (which arrives directly from first surface 152), such that the light exiting through first surface 152, indicated by light rays 720e , arrives close to focused at reference sensor 136 and, consequently, with high intensity. High intensity increases the signal-to-noise ratio. In particular, the high intensity may help to offset the contribution of scattered light, as well as light not originating from light source 122 which enters prism 110, to the obtained reference signal S2.

The power of the light incident on reference sensor 136 is substantially independent of n_{f}, as light rays 720 are not incident on any direct prism-fluid interface (e.g. second surface 154 and third surface 156). The power of the light incident on reference sensor 136 substantially equals the power of second sub-beam 420, and is therefore related by a fixed proportionality factor to light beam 400 power (i.e. the power of the light emitted by light source 122 and reflected by the mirror coating of fourth surface 158, which is limited by the clear aperture of fourth surface 158 mirror). As the power (and intensity) of light beam 400 may vary, e.g. due to changes in temperature or fluctuations in the driving current of light source 122 in embodiments wherein light source 122 is a LED, reference signal S2 is indicative of the power and the intensity of light beam 400. In particular, reference signal S2 may be used to “normalize” the signal generated by light sensor 124 (i.e. signal S1), and thereby to improve the measurement accuracy of n_{f}. That is to say, the ratio of S1 to S2 provides a measure indicative (up to a fixed proportionality factor) of the percentage of light—emitted from light source 122 and arriving at light sensor 124—that is impervious to (i.e. not affected by) fluctuations in light beam 400 power.

To facilitate the description, in FIG. 7A, only light rays on plane P are indicated. FIG. 7B presents a perspective view of prism 110. (Prism 110 is submerged in a fluid (not depicted). Light source 122 and reference sensor 136 are not shown.) The spread of second sub-beam 420 within prism 110 (beyond plane P), and travel-paths of light rays 720 within prism 110 (also outside of plane P), are depicted.

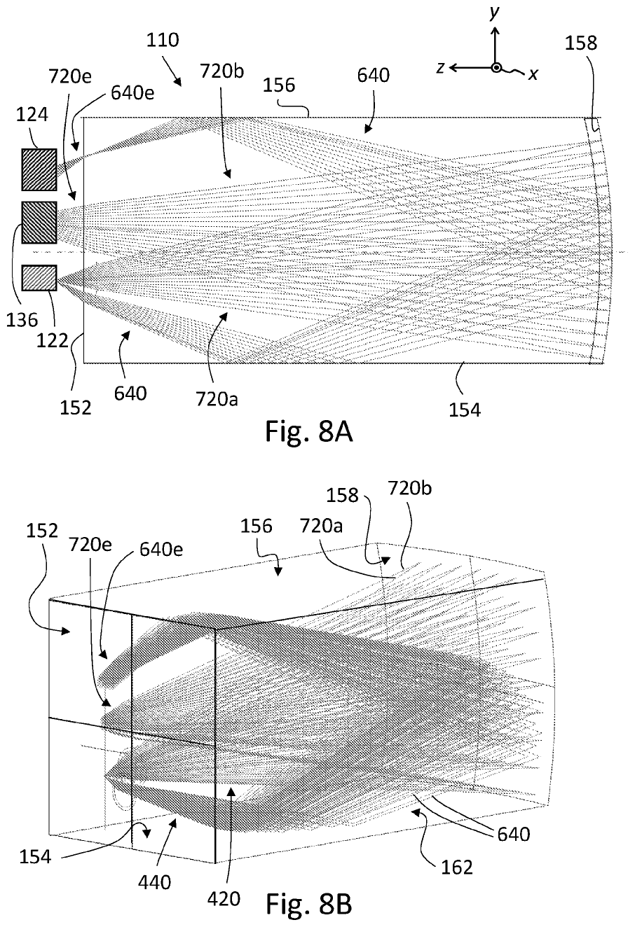

FIG. 8A combines FIG. 6A and FIG. 7A into a single figure. More specifically, FIG. 8A presents a cross-sectional view of prism 110 taken along plane P. Prism 110 is submerged in a fluid (not depicted). Light source 122 and sensors 124 and 136 are also shown. The travel-paths of light rays from both second sub-beam portion 440 and second sub-beam 420 are depicted.

FIG. 8B combines FIG. 6B and FIG. 7B into a single figure. More specifically, FIG. 8B presents a perspective view of prism 110. (Prism 110 is submerged in a fluid (not depicted). Light source 122 and sensors 124 and 136 are not shown.) The spreads of second sub-beam portion 440 and second sub-beam 420 within prism 110, and travel-paths of light rays 640 and 720 within prism 110 (also outside of plane P), are depicted.

The dependence of the spread (and power) of second-sub beam 440 on the fluid's refractive index (i.e. the dependence of the size of second incidence area 154b on n_{f}) is illustrated in FIGS. 9A-9F for a specific exemplary embodiment of refractometer 100 detailed below. FIGS. 9A-9F each presents a cross-sectional view of prism 110 taken along plane P. Prism 110 is submerged in a fluid (not depicted). Light source 122 and light sensors 124 are not shown. The travel-paths within prism 110 of light rays from second sub-beam portion 440, travelling on plane P, are depicted for n_{f}=1.33 (FIG. 9A), n_{f}=1.35 (FIG. 9B), n_{f}=1.37 (FIG. 9C), n_{f}=1.39 (FIG. 9D), n_{f}=1.41 (FIG. 9E), and n_{f}=1.43 (FIG. 9F).

In the exemplary specific embodiment, prism 110 is made of N-BK7 (or equivalent) glass having a refractive index nₚ=1.517. Prism 110 measures 12.5 mm in length and 6.0 mm in width and depth, and excepting the convexity of fourth surface 158, defines a rectangular box. Fourth surface 158 is spherical with a radius of curvature of 15.0 mm. Optical axis O is offset by 0.33 mm, relative to longitudinal symmetry axis A, towards second surface 154. Substrate bottom edge 212 is positioned at a distance of 0.70 mm from first surface 152 (i.e. gap G is 0.7 mm wide). (Light-emitting portion 122a (of light source 122), light-sensing surface 124a (of light sensor 124), and light-sensing surface 136a (of reference sensor 136) are each positioned on substrate bottom edge 212.)

Light emitting portion 122a is centered on plane P, 0.98 mm below longitudinal symmetry axis A. Light sensing surface 136a is centered on plane P, 0.32 mm above longitudinal symmetry axis A. Light sensing surface 124a is centered on plane P, 1.62 mm above longitudinal symmetry axis A. The numerical aperture of incoming light beam 400 measures approximately 0.62 mm along plane P and 0.34 mm on a plane parallel to the xy-plane (and perpendicular to plane P). Light beam 400 is substantially equally divided between the first incoming sub-beam (i.e. first sub-beam 410 prior to the entry thereof into prism 110) and the second incoming sub-beam (i.e. second sub-beam 420 prior to the entry thereof into prism 110). Light source 122 is a LED configured to emit light at 611 nm. Light sensors 124 and 136 are both phototransistors.

As seen in FIGS. 9A-9F, as n_{f} is increased, increasingly more light rays in first sub-beam 410 are refracted, and the spread (and consequently the power) of second sub-beam portion 440 decreases. For n_{f}=1.33, substantially no light rays in first sub-beam 410 are refracted (i.e. first sub-beam 410 consists of second sub-beam portion 440). For n_{f}=1.43, most of the light rays in first sub-beam 410 are refracted (i.e. the bulk of first sub-beam 410 is made up of first sub-beam portion 430 and third sub-beam portion 450). As illustrated in Table 1 and elaborated on below, for most of refractometer 100 measurement range, the bulk of contribution arises from second sub-beam portion 440. In the above-described specific exemplary embodiment, refractometer 100 measurement range extends between approximately 1.33 to approximately 1.45.

According to some embodiments, prism 110 may have a refractive index of 1.80 or even 2.00, thereby allowing measuring the refractive indices of fluids with high refractive indices.

As mentioned above, to facilitate the description, light source 122 was assumed to emit monochromatic light or light having a narrow spectral width. Nevertheless, the skilled person will appreciate that refractometer 100 function is not dependent on light source 122 emitting a monochromatic light beam or a light beam having a narrow spectral width. For example, light source 122 may be configured to emit light having a broad spectral width, e.g. white light. It is noted that when light source 122 emits light having a broad spectral width, the border between incidence areas on second surface 154 may not be sharp as each wavelength has associated therewith a respective critical angle. However, as refractometer 100 is configured to measure the overall power of light (originating from light source 122) incident on light sensor 124, a blurred or indistinct border (between the incidence areas on second surface 154) does not hinder refractometer 100 function, since for each wavelength the amount of light refracted by second surface 154 and third surface 156 (i.e. the amount of light transmitted to the fluid) increases monotonically with the refractive index of the fluid (though the rate of increase may depend on the wavelength).

According to some embodiments, light source 122 may be configured to emit a light beam including light of two distinct wavelengths: a first wavelength λ₁ and a second wavelength λ₂. Each of the two lights has associated therewith a respective critical angle (for given values of prism 110 refractive index, n_{f}, and temperature). Consequently, the λ₁ light and λ₂ light are “sensitive” to a first range of fluid refractive indices and a second range of fluid refractive indices, respectively, as follows: An amount of λ₁ light (emitted by light source 122) reflected off second surface 154 varies with n_{f} when n_{f} is in the first range, but is substantially constant when n_{f} is in the second range. An amount of λ₂ light (emitted by light source 122) reflected off second surface 154 varies with n_{f} when n_{f} is in the second range, but is substantially constant when n_{f} is in the first range.

For instance, the first range and second range may be complementary with the first range ranging from 1.33 (the refractive index of water at room temperature) to 1.41, and the second range ranging from 1.41 to 1.49. When n_{f}=1.33, substantially all of the λ₁ light, emitted by light source 122 and incident on second surface 154, undergoes TIR off second surface 154. As n_{f} is increased beyond 1.33, the λ₁ light starts refracting on second surface 154, with the amount of refracted λ₁ light increasing with n_{f} until no light rays of the first wavelength undergo TIR off second surface 154 when n_{f}=1.41. λ₂ light, emitted by light source 122 and incident on second surface 154, undergoes TIR off second surface 154 for 1.33≤n_{f}≤1.41. As n_{f} is increased beyond 1.41, the λ₂ light starts refracting on second surface 154, with the amount of refracted λ₂ light increasing with n_{f} until no light rays of the second wavelength undergo TIR off second surface 154 when n_{f}=1.49.

FIG. 10 depicts a block-diagram of refractometer 100, according to some embodiments. Optional elements (such as temperature sensor 138) are represented by boxes outlined by dashed lines (as opposed to non-optional elements which are represented by boxes outlined by solid lines). Control unit 130 includes electronic circuitry in the form of processing circuitry 1010 (CPU and memory circuitry). Processing circuitry 1010 may include application specific integrated circuitry (ASIC), a programmable processing circuitry such as an FPGA, firmware, and/or the like. Control unit 130 further includes power-supply circuitry (not shown) coupling battery 132 to processing circuitry 1010 and refractometer 100 components requiring external powering (e.g. light source 122, as opposed to temperature sensor 138 in embodiments including temperature sensor 138 and wherein temperature sensor 138 is a thermocouple).

Processing circuitry 1010 is electrically coupled (e.g. via electrical wirings, not shown) to light sensor 122 and is configured to control the operation thereof, e.g. switch on light sensor 122 to initiate a measurement of the fluid's refractive index n_{f} Processing circuitry 1010 has stored in the memory circuitry (e.g. a flash memory) dedicated software for processing sensors 124, 136, and 138 respective outputs (i.e. electrical signals S1, S2, S3) to obtain the value of n_{f} and/or optionally the concentration of a tastant in the fluid or the concentrations of a number of tastants in the fluid.

Electrical signal S1 is indicative of the power of the light incident on light sensor 124. To obtain n_{f}, processing circuitry 1010 relates the power of the light incident on light sensor 124 to the power of first sub-beam 410 (i.e. the light incident on second surface 154). However, the power of first sub-beam 410 fluctuates with the power of light beam 400 (i.e. the output of light source 122). Electrical signal S2, being substantially proportional to second sub-beam 420 power, is indicative of light beam 400 power, thereby allowing processing circuitry 1010 to factor in light beam 400 power fluctuations. More specifically, the ratio of S1 to S2 (or of amplified signals obtained therefrom, respectively) provides a measure that is indicative (up to a fixed proportionality constant) of the percentage of light beam 400 light which arrives at light sensor 124, the advantage of the measure being that that the ratio is unaffected by fluctuations in light beam 400 power. Finally, as n_{f} is dependent on the temperature of prism 110, and as electrical signal S3 is indicative of the temperature of the fluid and therefore the temperature of prism 110 (when the fluid and prism 110 reach thermal equilibrium), electrical signal S3 allows processing circuitry 1010 to take into account the temperature of prism 110 in computing n_{f}.

Electrical signals S1, S2, and S3 may undergo initial (individual) processing prior to being fed into processing circuitry 1010. For example, control unit 130 may further include amplifiers (not shown), e.g. for amplifying electrical signals S1 and S2 prior to being fed into processing circuitry 1010. And one or more convertors (not shown), e.g. an analog-to-digital (A/D) convertor for converting electrical signal S3 into a digital signal and/or a resistance-to-voltage (R/V) convertor in embodiments wherein temperature sensor 138 is a RTD.

According to some embodiments, processing circuitry 1010 is configured to compute n_{f} only after the sensor readings (i.e. signals S1, S2, and S3) have stabilized. In particular, stability of signals S1 and S2 may indicate that prism 110 has reached thermal equilibrium with the fluid. The computation may involve averaging over time. That is to say, averaging over n_{f} values, each value obtained from signals S1, S2, and S3 corresponding to a distinct sampling intervals (time-intervals).

It is noted that n_{f} may be computed from light sensor 124 signal S1 and reference sensor 136 signal S2 without taking into account a temperature reading (such as temperature sensor 138 signal S3). In particular, according to some embodiments wherein refractometer 100 does not include temperature sensor 138, processing circuitry 1010 is configured to compute n_{f} from signals S1 and S2.

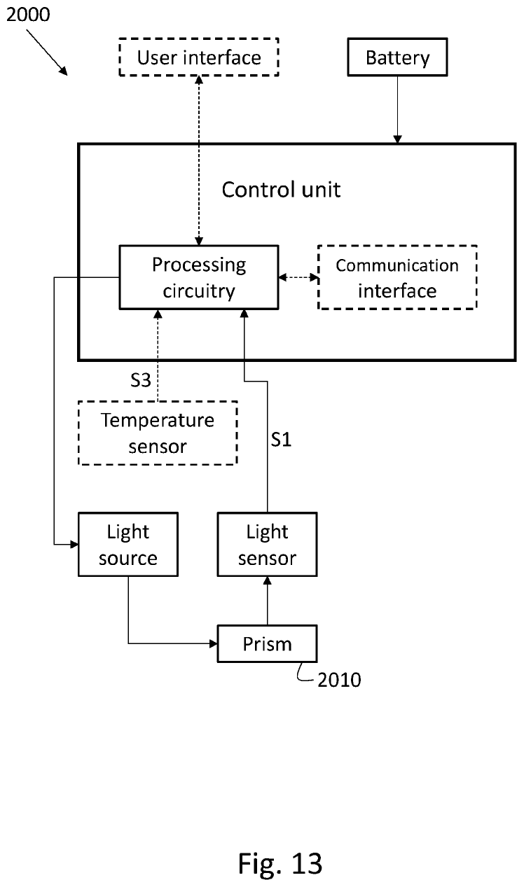

It is also noted that n_{f} may be computed from light sensor 124 signal S1 and temperature sensor 138 signal S3 without taking into account reference sensor 136 signal S2. Specifically, according to some embodiments, as elaborated on below in the description of FIG. 13, the refractometer does not include a reference sensor, such as reference sensor 136, and n_{f} is computed from signal S1, or from signal S1 and from the measured temperature of the prism (e.g. from signal S3) in embodiments including a temperature sensor.

According to some embodiments, control unit 130 further includes a communication interface 1020 configured for wireless communication (e.g. Bluetooth or Wi-Fi) with an external device, such as a smartphone, a personal computer, an online server, and/or the like. Communication interface 1020 allows sending obtained measurement data to the external device. According to some such embodiments, the computation of n_{f} and/or the concentration of a tastant may be carried out on the external device. According to some embodiments, dedicated software installed on the external device, e.g. a dedicated app on installed on a smartphone, is configured to allow a user to control/partially control refractometer 100 operation (e.g. instruct refractometer 100 to start measuring). In some such embodiments, refractometer 100 does not include user interface 134. According to some embodiments, communication interface 1020 may additionally/alternatively be configured for wired communication with an external device. In such embodiments, refractometer 100 includes a port, e.g. a micro USB port, allowing for wired data transfer to/from an external device, such as a smartphone or a tablet.

According to some embodiments, updates to processing circuitry 1010 software may be downloaded from an online server via communication interface 1020. The updates may include new or improved data relating refractive indices of fluids to the respective concentrations of tastants therein.

Table 1 presents results of calculation of intensities of light rays, originating from light source 122, as a function of the incidence angles thereof on second surface 154 and third surface 156, and as a function of n_{f}—the refractive index of the fluid. The calculation is based on the Fresnel equations for the reflection and refraction of light at the interface between two media, and was carried out with respect to a specific exemplary embodiment of prism 110, wherein prism 110 geometry is symmetrical and consequently the path of a light ray before and after reflection off fourth surface 158 is symmetrical. That is to say: (i) optical axis O coincides with longitudinal symmetry axis A, so that the sum of respective incidence angles θ₂ and θ₃ on second surface 154 and third surface 156, respectively, of each light ray originating from first beam 410 equals a constant c; and (ii) the emission point and the focusing point of light rays emitted by light source 122 that are reflected off second surface 154 (i.e. the center-point of light-emitting portion 122a and the center-point of light sensing portion 124a , respectively), are fully symmetrical relative to optical axis O (and longitudinal symmetry axis A) of prism 110.

More specifically, given that a light ray, e.g. light ray 530, traces a path within prism 110 such that the incidence angle thereof on second surface 154 equals θ₂, then the incidence angle thereof on third surface 156 will equal θ₃=c−θ₂. In particular, to each light ray incident on second surface 154 and third surface 156 at angles angle θ₂ and θ₃, respectively, there corresponds another light ray incident on second surface 154 and third surface 156 at angles θ₃ and θ₂, respectively.

Light source 122 emits light having a wavelength of 611 nm. The fluid submerging prism 110 is at a temperature of 20° C. Prism 110 is made of N-BK7 (or equivalent) glass having a refractive index nₚ=1.517. Prism 110 is rectangular, having a length of 12.50 mm and a square (transverse) cross-section measuring 6.00 mm×6.00 mm. Fourth surface 158 is spherical with a radius of curvature of 15.80 mm. Light-emitting portion 122a and light-sensing surface 124a are each centered on plane P, 0.67 mm below and 0.67 mm above longitudinal symmetry axis A (and optical axis O), at a distance of 0.50 mm from first surface 152. The numerical aperture of the incoming light beam measures approximately 0.62 mm along plane P and 0.34 mm on a plane parallel to the xy-plane (and perpendicular to plane P).

As seen in Table 1, the contributions of both first sub-beam portion 430 and third sub-beam portion 450 to the power, detected by light sensor 124, are marginal, as compared to the contribution of second sub-beam portion 440, except for values of n_{f} close to the top of the measurement range of refractometer 100—the measurement range being approximately 1.33-1.45. Consequently, as compared to an alternative refractometer (not depicted in the figures), identical to refractometer 100 in all respects except that the third surface of the prism of the alternative refractometer includes a mirror coating (and therefore does not form a direct prism-fluid interface with the fluid), the measurement resolution of refractometer 100 is substantially twice as high. This last point is illustrated in FIG. 11. A curve C1 shows the (normalized) power P_{N} incident on light sensor 124 as a function of n_{f} A curve C2 shows the (normalized) power incident on the light sensor of the alternative refractometer as a function of n_{f} The slope of C1 is substantially twice as steep as the slope of C2 (i.e. for each value of n_{f} in the range, the derivative of C1 is substantially double that of C2). As seen in FIG. 11, for any two close values of n_{f} within the measurement range (1.33-1.45), the difference of the respective powers of the light beams incident on light sensor 124 is substantially higher than the difference of the respective powers of the light beams incident on the light sensor of the alternative refractometer, implying substantially higher measurement resolution. In particular, for curve C1, at n_{f}=1.33 and n_{f}=1.45 P_{N} equals 1.00 and 0.05, respectively, corresponding to a 20:1 ratio, while, in comparison, for curve C2 the corresponding ratio is only 2:1.

To facilitate the description, the above-described calculation was carried out with respect a symmetrically-configured embodiment of prism 110. However, the skilled person will appreciate that a similar increase in the measurement resolution, relative to the alternative refractometer described above, may also obtained in non-symmetrically configured embodiments of prism 110, e.g. wherein optical axis O is slightly offset with respect to longitudinal symmetry axis A.

TABLE 1

| | n_{f} = 1.33 | n_{f} = 1.35 | n_{f} = 1.37 | n_{f} = 1.39 | n_{f} = 1.41 | n_{f} = 1.43 | n_{f} = 1.45 |
|---|---|---|---|---|---|---|---|
| θ₂ = 62°, θ₃ = 82° | 1.000 | 0.291 | 0.114 | 0.054 | 0.027 | 0.014 | 0.006 |
| θ₂ = 64°, θ₃ = 80° | 1.000 | 1.000 | 0.364 | 0.121 | 0.053 | 0.024 | 0.011 |
| θ₂ = 66°, θ₃ = 78° | 1.000 | 1.000 | 1.000 | 0.429 | 0.118 | 0.047 | 0.019 |
| θ₂ = 68°, θ₃ = 76° | 1.000 | 1.000 | 1.000 | 1.000 | 0.466 | 0.107 | 0.037 |
| θ₂ = 70°, θ₃ = 74° | 1.000 | 1.000 | 1.000 | 1.000 | 1.000 | 0.365 | 0.079 |
| θ₂ = 72°, θ₃ = 72° | 1.000 | 1.000 | 1.000 | 1.000 | 1.000 | 1.000 | 0.059 |
| θ₂ = 74°, θ₃ = 70° | 1.000 | 1.000 | 1.000 | 1.000 | 1.000 | 0.365 | 0.079 |
| θ₂ = 76°, θ₃ = 68° | 1.000 | 1.000 | 1.000 | 1.000 | 0.466 | 0.107 | 0.037 |
| θ₂ = 78°, θ₃ = 66° | 1.000 | 1.000 | 1.000 | 0.429 | 0.118 | 0.047 | 0.019 |
| θ₂ = 80°, θ₃ = 64° | 1.000 | 1.000 | 0.364 | 0.121 | 0.053 | 0.024 | 0.011 |
| θ₂ = 82°, θ₃ = 62° | 1.000 | 0.291 | 0.114 | 0.054 | 0.027 | 0.014 | 0.006 |

It is noted that in a final stage of production of refractometer 100, refractometer 100 may be calibrated by performing measurements on various fluids at different temperatures—the refractive indices of the fluids having a known dependence on the temperature—to verify, and if need be, adjust the dependence (encoded in processing circuitry 1010 software) of the computed refractive index on sensors 124, 136, and 138 signals, i.e. S1, S2, and S3.

According to some embodiments, light source 122 includes a number of LEDs, e.g. two LEDs, three LEDs, or even five LEDs. Each LED is configured to emit light having a unique peak wavelength. Each peak wavelength corresponds to a respective measurement range of n_{f}, thereby increasing the overall refractive index measurement range of refractometer 100.

According to some embodiments, the numerical aperture of light beam 400 (or of the light-beams emitted by each of the LEDs, respectively, in embodiments wherein light source 122 includes more than one LED) is controllably modifiable, for example, light source 122 may include a controllable shutter (not shown). The measurement accuracy and/or the measurement range can thereby be increased. In particular, the numerical aperture can be increased or decreased according to the temperature of the fluid.

According to some embodiments (not depicted in the figures), refractometer 100 is installed in a kitchen utensil, such as a cooking pot or a cocktail shaker, e.g. on an inner surface thereof.

According to some embodiments, the measured concentration of a tastant may be displayed in e.g. g/L (grams per liter) on display 142. According to some embodiments, the measured concentration of a tastant may be displayed in Val units on display 142. As disclosed in PCT Pub. No. WO 2015/011698 to Klein, the Val scale is a universal scale to quantify magnitudes, e.g. concentrations of tastants, and perceptions, e.g. flavor perceptions. For example, Val sweetness quantifies a concentration of sugars, while Val sourness quantifies a concentration of acids. The Val scales are calibrated such that 1 Val marks a threshold where the average person will start sensing a respective flavor in standard conditions of otherwise (i.e. except for a presence of the respective tastant) clear water at 20° C. Thus, 1 Val sweetness corresponds to a sucrose concentration of 3.42 g/L in otherwise clear water at 20° C.

According to some embodiments, light source 122 is configured to emit/additionally emit light outside the visible spectrum, such as infrared light or ultraviolet light. According to some such embodiments, light sensor 124 and reference sensor 136 are sensitive/also sensitive to light outside the visible spectrum.

As used herein, “reference sensor” and “reference light sensor” are interchangeable.

FIG. 12 depicts a cross-sectional view of a bottom part of a dipping refractometer 1200 submerged in a fluid (not shown). Refractometer 1200 includes a casing 1202, of which only a bottom part of an immersion portion 1206 thereof is shown, and a triangular prism 1210. Refractometer 1200 is similar to refractometer 100 but differs therefrom as elaborated on below, particularly in prism 1210 being triangular and in an arrangement of sensors in immersion portion 1206.

Triangular prism 1210 includes a first surface 1252, a second surface 1254, and a third surface 1256. Triangular prism 1210 is mounted on immersion portion 1206 bottom such that second surface 1254 is exposed. Second surface 1254 includes a first area 1254a and a second area 1254b . First area 1254a forms a direct prism-fluid interface when immersion portion 1206 is submerged in a fluid. Second area 1254b is coated by a mirror coating.

A light sensor 1224 and a reference light sensor 1236, similar to light sensor 124 and reference light sensor 136, respectively, are positioned above third surface 1256, each being configured to send to a control unit (not shown), such as control unit 130, a respective signal indicative of a respective power of light incident thereon (similarly to sensors 124 and 136 respective signals S1 and S2).

A light source system 1222 is positioned opposite first surface 1252. Light source system 1222 may include a light source 1222a (e.g. a LED) and a means 1222b (e.g. a beam-splitter and a pair of mirrors) for splitting a light beam 1400 emitted from light source 1222a into a first sub-beam 1410 and a second sub-beam 1420, such that first sub-beam 1410 and second sub-beam 1420 are incident on first surface 1252 and enter triangular prism 1210 there through. Prism 1210 and light source system 1222 are configured such that second sub-beam 1420 is directed onto second area 1254b and is reflected therefrom toward third surface 1256, exiting through third surface 1256 such as to be incident on reference light sensor 1236.

Prism 1210 and light source system 1222 are further configured such that first sub-beam 1410 is directed onto first area 1254a . First sub-beam 1410 includes two sub-beam portions: a first sub-beam portion 1430 and a second sub-beam portion 1440. Each of the light rays in first sub-beam portion 1430 is incident on first area 1254a at a respective angle smaller than a critical angle defined by the direct prism-fluid interface and is mostly refracted into the fluid (not shown). Each of the light rays in second sub-beam portion 1440 is incident on area 1254a , at a respective angle greater than the critical angle defined by the direct prism-fluid interface, and undergoes TIR being reflected towards third surface 1256 and exiting there through such as to be incident on light sensor 1224.

Making reference to FIG. 13, according to some embodiments, there is provided a dipping refractometer 2000. FIG. 13 depicts a block diagram of refractometer 2000. Optional elements (such as temperature sensor 138) are represented by boxes outlined by dashed lines (as opposed to non-optional elements which are represented by boxes outlined by solid lines). Refractometer 2000 includes a casing (not depicted in the figures) similar to casing 102 and a prism 2010, mounted on the casing similarly to prism 110 mounting on casing 102. Prism 2010 is similar to prism 110 embodiments which include at least two exposed surfaces (that form two respective direct prism-fluid surfaces when refractometer 2000 is dipped in a fluid).

It is noted that refractometer 2000 differs from refractometer 100 in not including a reference sensor, such as reference sensor 136. The light source is configured such that substantially all of the light emitted therefrom is incident (after entry into prism 2010) on the second surface of prism 2010 (corresponding to second surface 154 of prism 110). That is to say, the light beam entering prism 2010 does not include a sub-beam, such as second sub-beam 420, which is incident on the fourth surface of prism 2010 (corresponding to fourth surface 158 of prism 110) without having first been reflected off the second surface.

According to an aspect of some embodiments, there is provided a dipping refractometer (e.g. refractometer 100). The dipping refractometer includes:
a casing, housing a light source, a light sensor, and a control unit; and
a prism (e.g. prism 110) including at least two exposed surfaces (e.g. second surface 154 and third surface 156);

The control unit includes electronic circuitry functionally associated with the light source and the light sensor. The prism is mounted in/on the casing such as to allow dipping the prism in a fluid with the exposed surfaces and the fluid forming respective direct prism-fluid interfaces. The prism, the light source, and the light sensor, are configured such that at least some of the light emitted from the light source enters the prism, travels to one exposed surface (e.g. second surface 154) and reflects therefrom, travels to the other exposed surface (e.g. third surface 156) and reflects therefrom, and travels to the light sensor. The light sensor is configured to send to the control unit a signal indicative of a power of a light incident on the light sensor.

According to some embodiments of the dipping refractometer, the dipping refractometer further includes a reference light sensor. The prism, the light source, and the reference light sensor are configured such that some of the light emitted by the light source travels through the prism without reflecting off either of the exposed surfaces (e.g. second surface 154 and third surface 156), exiting the prism such as to be incident on the reference light sensor. The reference light sensor is further configured to send to the control unit a reference signal, indicative of a power of the light incident thereon.

According to some embodiments of the dipping refractometer, substantially all the light incident on the light sensor, which originates from the light source, is reflected by both of the exposed surfaces (e.g. second surface 154 and third surface 156) when travelling through the prism.

According to some embodiments of the dipping refractometer, the prism includes a light entry surface (e.g. first surface 152) where through light emitted from the light source enters the prism and where through the light incident on the light sensor exits the prism.

According to some embodiments of the dipping refractometer, the prism further includes a reflective surface (e.g. fourth surface 158) including a mirror coating. The prism, the light source, and the light sensor are further configured such that light emitted from the light source, which is incident on one exposed surface (e.g. second surface 154), reflects from the exposed surface to the reflective surface, and reflects from the reflective surface to the other exposed surface (e.g. third surface 156), travelling therefrom to the light sensor.

According to some embodiments of the dipping refractometer, the reflective surface (e.g. fourth surface 158) is located opposite the light entry surface (e.g. first surface 152), and the exposed surfaces (e.g. second surface 154 and third surface 156) are located opposite to one another. The exposed surfaces extend from the light entry surface to the reflective surface.

As used herein, according to some embodiments, the terms “incident” and “impinging” are interchangeable.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. No feature described in the context of an embodiment is to be considered an essential feature of that embodiment, unless explicitly specified as such.

Although steps of methods according to some embodiments may be described in a specific sequence, methods of the invention may comprise some or all of the described steps carried out in a different order. A method of the invention may comprise all of the steps described or only a few of the described steps. No particular step in a disclosed method is to be considered an essential step of that method, unless explicitly specified as such.

Although the invention is described in conjunction with specific embodiments thereof, it is evident that numerous alternatives, modifications and variations that are apparent to those skilled in the art may exist. Accordingly, the invention embraces all such alternatives, modifications and variations that fall within the scope of the appended claims. It is to be understood that the invention is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the invention. Section headings are used herein to ease understanding of the specification and should not be construed as necessarily limiting.

1.-40. (canceled)

41. A dipping refractometer, comprising:
a casing, housing a light source, a light sensor, and a control unit; and
a prism comprising at least two exposed surfaces;
wherein said control unit comprises electronic circuitry functionally associated with said light source and said light sensor;
wherein said prism is mounted in or on said casing and allowing to dip said prism in a fluid such that said exposed surfaces and the fluid forming respective direct prism-fluid interfaces;
wherein said prism, said light source, and said light sensor, are configured such that at least some of the light emitted from said light source enters said prism, travels to one exposed surface and reflects therefrom, travels to the other exposed surface and reflects therefrom, travels to said light sensor; and
wherein said light sensor is configured to send to said control unit a signal indicative of a power of a light incident on said light sensor.

42. The refractometer of claim 41, further comprising a temperature sensor configured to measure the temperature of said prism and send a second signal to said control unit indicative said temperature measurement.

43. The refractometer of claim 41, further comprising a reference light sensor, wherein said prism, said light source, and said reference light sensor are configured such that some of the light emitted by said light source travels through said prism without reflecting off either of said exposed surfaces and exiting said prism such as to be incident on said reference light sensor, said reference light sensor being further configured to send to said control unit a reference signal, indicative of a power of the light incident on said reference light sensor.

44. The refractometer of claim 41, wherein substantially all the light incident on said light sensor, which originates from said light source, is reflected by both of said exposed surfaces when travelling through said prism.

45. The refractometer of claim 41, wherein said prism comprises a light entry surface where through light emitted from said light source enters said prism and where through the light incident on said light sensor exits said prism.

46. The refractometer of claim 45, wherein said prism further comprises a reflective surface comprising a mirror coating; said prism, said light source, and said light sensor being further configured such that light emitted from said light source, which is incident on one exposed surface, reflects from said exposed surface to said reflective surface, and reflects from said reflective surface to the other exposed surface, travelling therefrom to said light sensor.

47. The refractometer of claim 46, wherein said reflective surface is located opposite to said light entry surface, and said exposed surfaces are located opposite to one another, said exposed surfaces extending from said light entry surface to said reflective surface.

48. The refractometer of claim 47, wherein said reflective surface is convex, being configured to function as a concave mirror with respect to light incident thereon from within said prism; said prism, said light source, and said light sensor being configured such that light exiting said prism, emitted by said light source and incident on said light sensor, is focused by said reflective surface such as to arrive with a small beam spread at said light sensor.

49. The refractometer of claim 48, wherein said prism comprises a rectangular prism and a spherical plano-convex lens mounted on a bottom surface of said rectangular prism, said plano-convex lens having a same refractive index as said rectangular prism, and wherein an optical axis defined by said plano-convex lens is offset relative to a longitudinal symmetry axis of said rectangular prism.

50. The refractometer of claim 41, wherein said light source is configured to emit monochromatic or polychromatic light.

51. The refractometer of claim 41, wherein said light source is a light-emitting diode or a laser diode.

52. The refractometer of claim 46, further configured such that the light received by said reference light sensor, which was emitted by said light source, enters said prism through said light entry surface, travels directly therefrom to said reflective surface, reflects therefrom back to said light entry surface and travels therefrom to said reference light sensor.

53. The refractometer of claim 41, wherein said electronic circuitry includes processing circuitry configured to determine a refractive index of a fluid, in which the refractometer is dipped, based on the signal received from said light sensor.

54. The refractometer of claim 53, wherein said processing circuitry is further configured to determine the refractive index of the fluid based on the second signal received from said temperature sensor, on the reference signal received from said reference light sensor, or based on both.

55. The refractometer of claim 53 , wherein said processing circuitry is configured to obtain a concentration of sugar in the fluid from the signals received from said sensors.

56. The refractometer of claim 41, wherein said casing is waterproof, wherein said casing is elongated, comprising an upper portion and an immersible lower portion, such as to allow said refractometer to be dipped in a fluid-filled drinking vessel and to be configured with a user interface on said upper portion being located above the fluid, said user interface being functionally associated with said control unit.

57. A method for determining the refractive index of a fluid, comprising the steps of:
submerging a prism in a fluid such that one or more surfaces of the prism form with the fluid one or more direct prism-fluid interfaces, respectively;
projecting a light beam into the prism;
directing at least some of the light in the light beam onto at least one of the one or more direct prism-fluid interfaces, and reflecting the light therefrom;
directing the reflected light onto at least one of the one or more direct prism-fluid interfaces, and reflecting the light therefrom;
directing the doubly-reflected light out of the prism and onto a light sensor;
converting the light arriving at the light sensor into an electrical signal indicative of the power of the arriving light; and
determining the refractive index of the fluid based on the obtained electrical signal.

58. The method of claim 57, further comprising a step of measuring a temperature of the prism, wherein the refractive index of the fluid is determined while taking into account also the measured temperature of the prism.

59. The method of claim 57, further comprising a step of measuring a power of light in the light beam, which is not directed onto any of the direct prism-fluid interfaces, thereby recording fluctuations in the power of the light beam, wherein the refractive index of the fluid is determined taking into account also the recorded fluctuations in the power of the light beam.

60. A dipping refractometer, comprising:
a casing, housing a light source and a light sensor; and
a prism;
wherein said prism is mounted in or on said casing such as to allow dipping said prism in a fluid with one or more surfaces of said prism and the fluid forming one or more direct prism-fluid interfaces, respectively;
wherein said prism, said light source, and said light sensor are configured such that for a continuous range of values of fluid refractive indices, most of the light incident on said light sensor, having travelled through said prism and originating from said light source, has undergone total internal reflection off the one or more direct prism-fluid interfaces at least twice.
